(19) United States
(12) Patent Application Publication
(10) Pub. No.: US 2003/0216594 A1
(43) Pub. Date: Nov. 20, 2003

(54) Process for the preparation of substituted crotonic acid esters

(76) Inventors: Reinhard Georg Hanreich, Basel (CH); Herng-Tay Wu, Baton Rouge, LA (US); Michael Allen Oliver, Daphne, AL (US); Dean Kent Hoglen, Baton Rouge, LA (US)

Correspondence Address: William A. Teoli, Jr., Syngenta Crop Protection, Inc., Patent and Trademark Dept., 410 Swing Road, Greensboro, NC, 27409, US

(21) Appl. No.: 09/878,661

(22) Filed: Mar. 23, 2001

Related U.S. Application Data
(63) Continuation of application No. 09/042,122, filed on Mar. 13, 1998, now abandoned.
(60) Provisional application No. 60/040,696, filed on Mar. 13, 1997.

(51) Int. Cl. C07C 229/20

(52) U.S. Cl. 560/172; 570/123; 570/124; 570/134; 570/135; 570/136

(57) ABSTRACT

According to the invention there is proposed a process for the preparation of 4,4,4-trifluoro-3-methylamino-crotonic acid esters by dehydrating trifluoroacetoacetic acid esters and methylamine in the presence of an inert reaction medium and of a C₁-C₄carboxylic acid, in which process the reaction is carried out at a temperature of from 75 to 100° C. without isolation of intermediates and the water that forms during the reaction is removed continuously from the reaction medium.

The present invention relates to a novel process for the preparation of 4,4,4-trifluoro-3-methylamino-crotonic acid ethyl esters by reaction of trifluoroacetoacetic acid esters with methylamine.

4,4,4-Trifluoro-3-methylamino-crotonic acid esters are valuable intermediates for the preparation of herbicidally active 3-phenyluracils, as are described, for example, in U.S. Pat. No. 5,183,492.

It is known from JP 05140060-A to prepare 4,4,4-trifluoro-3-methylamino-crotonic acid ethyl esters as follows:

a) trifluoroacetoacetic acid ethyl ester is reacted with methylamine in the presence of a solvent to form the ammonium salt

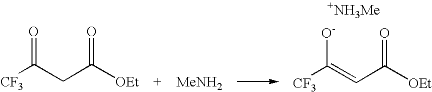

b) the ammonium salt so obtained is isolated, and

c) is then dehydrated in the presence of an organic solvent and of a C₁-C₄carboxylic acid to form the 4,4,4-trifluoro-3-methylamino-crotonic acid ethyl ester.

The yields of only 63% 4,4,4-trifluoro-3-methylamino-crotonic acid ethyl ester achieved with that process are, however, very uneconomic and, for large-scale applications in particular, are completely inadequate.

Surprisingly, it has now been found that the yields in such reactions can be increased considerably if the reaction is carried out in a single step without isolation of the ammonium salt, with simultaneous removal of the water that forms, at a temperature that is suited specifically to the reaction procedure.

According to the invention, therefore, there is proposed a process for the preparation of 4,4,4-trifluoro-3-methylamino-crotonic acid esters of formula I

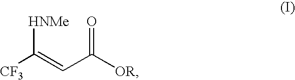

wherein

R is C₁-C₄alkyl, and

Me is methyl,

by dehydrating the corresponding trifluoroacetoacetic acid ester and methylamine in the presence of an inert reaction medium and of a C₁-C₄carboxylic acid, in which process the reaction is carried out at a temperature of from 75 to 100° C. without isolation of intermediates and the water that forms during the reaction is removed continuously from the reaction medium.

C₁-C₄Alkyl is methyl, ethyl, propyl, isopropyl, butyl, isobutyl, sec-butyl or tert-butyl.

The process according to the invention is especially suitable for the preparation of 4,4,4-trifluoro-3-methylamino-crotonic acid ethyl ester.

In a preferred variant of the process according to the invention, a water-immiscible inert reaction medium is used. By removing the water of reaction that forms from the organic phase, the undesired hydrolysis of the starting material, or product, can be kept to a low level. At the end of the reaction, the water of reaction separates as the lower phase and can therefore be separated from the upper, organic product phase in a simple manner.

The desired product is then obtained by fractional distillation of the organic phase.

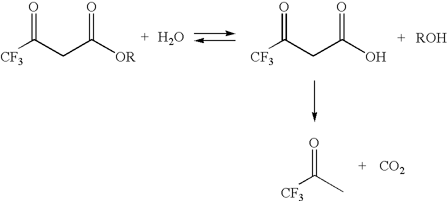

In another preferred variant of the process according to the invention, a reaction medium is used to which there is added the appropriate alcohol (for example, in the preparation of 4,4,4-trifluoro-3-methylamino-crotonic acid ethyl ester: ethanol) in order to shift the hydrolysis equilibrium to the side of the starting material.

The water of reaction is removed continuously during the reaction by azeotropic distillation. The alcohol content in the reaction medium is from 15% by weight to 40% by weight, preferably from 25 to 30% by weight, based on the reaction medium used.

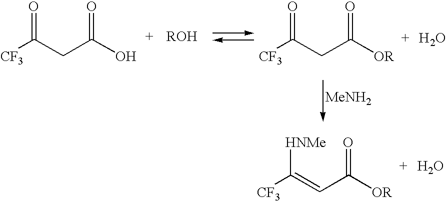

In this variant, the distillation fraction (water/alcohol) is discarded and the solvent fraction (solvent/alcohol) is recycled. The distillation is customarily carried out under atmospheric pressure. When the distillation is complete, the excess alcohol is removed by fractional distillation in vacuo. The pressure is advantageously so selected that the temperature of the reaction mixture does not exceed 100° C. When the distillation is complete and after cooling to room temperature, a two-phase mixture is obtained. The lower phase, which contains the carboxylic acid used as well as methylammonium acetate, can be separated off in a simple manner. The desired product is then obtained by fractional distillation of the organic phase.

Acetic acid is preferably used as the C₁-C₄carboxylic acid. From 0.25 to 0.3 equivalent of acetic acid, based on the trifluoroacetoacetic acid ethyl ester employed, is generally used.

A preferred temperature range for the process according to the invention is from 80 to 85° C. Especially suitable inert reaction media are hexane, cyclohexane, methylcyclohexane, toluene and xylene, and mixtures thereof. The use of methylcyclohexane is especially advantageous.

Methylamine is used in gaseous form in an amount of from 1.05 to 1.2 equivalents (based on trifluoroacetoacetic acid ester). 1.1 equivalents are preferred. The metered addition of the methylamine is carried out in such a manner that the ammonium salt that forms in the reaction medium as an intermediate goes continuously into solution.

In the process according to the invention, as a result of the specific selection of the reaction conditions, the ammonium salt is formed in situ and reacted directly to form the 4,4,4-trifluoro-3-methylamino-crotonic acid ester, the water that forms during the reaction being removed continuously from the reaction medium. The one-step process according to the invention allows the 4,4,4-trifluoro-3-methylamino-crotonic acid ester to be prepared simply and economically in considerably higher yields than those described in the mentioned prior art. As well as avoiding the time-consuming and, especially from the point of view of large-scale application, completely uneconomic isolation of the ammonium salt as in the three-step process, the one-step process according to the invention achieves surprisingly high yields of up to 98% of the theoretical yield.

The Examples which follow illustrate the invention in greater detail without limiting it.

### EXAMPLE P1

### Preparation of 4.4.4-trifluoro-3-methylamino-crotonic Acid Ethyl Ester

28 g (0.47 mol) of acetic acid are added to a solution of 280 g (1.49 mol) of trifluoroacetoacetic acid ethyl ester in 300 g of methylcyclohexane, and the mixture is heated to a temperature of 80-85° C. Then 58 g (1.87 mol) of gaseous methylamine are introduced within a period of 6 hours in such a manner that the ammonium salt that forms goes continuously into solution without precipitating. After maintaining that temperature for 2 hours, the mixture is cooled to a temperature of 20-25° C., the lower, acetic acid/water phase is separated off and the upper, organic phase is fractionated. There are obtained 270 g (90% of the theoretical yield) of 4,4,4-trifluoro-3-methylamino-crotonic acid ethyl ester having a boiling point of 95° C. (product fraction 95° C./25 mbar).

### EXAMPLE P2

### Preparation of 4.4.4-trifluoro-3-methylamino-crotonic Acid Ethyl Ester

12 g (0.2 mol) of acetic acid are added to a solution of 112 g (0.61 mol) of trifluoroacetoacetic acid ethyl ester in 117 g of methylcyclohexane, and the mixture is heated to a temperature of 80-85° C. Then 13 g (0.74 mol) of gaseous methylamine are introduced within a period of 2 hours, with stirring, in such a manner that the ammonium salt that forms goes continuously into solution without precipitating. After maintaining that temperature for one hour, the mixture is cooled to a temperature of 20° C., the lower, acetic acid/water phase is separated off and the upper, organic phase is fractionated. There are obtained 102 g (88.5% of the theoretical yield) of 4,4,4-trifluoro-3-methylamino-crotonic acid ethyl ester having a boiling point of 95-98° C. (product fraction 95-98° C./25 mbar).

### EXAMPLE P3

### Preparation of 4.4.4-trifluoro-3-methylamino-crotonic Acid Ethyl Ester

32 g (0.53 mol) of acetic acid are added to a solution of 400 g (2.17 mol) of trifluoroacetoacetic acid ethyl ester in 420 g of methylcyclohexane and 240 g of ethanol, and the mixture is heated to a temperature of 80-85° C. Then 71 g (2.29 mol) of gaseous methylamine are introduced within a period of 3 to 4 hours, with stirring, in such a manner that the ammonium salt that forms goes continuously into solution without precipitating. During the addition of the methylamine, the water that forms is removed continuously from the reaction mixture by azeotropic distillation under atmospheric pressure. If the reaction temperature exceeds 90° C. during the distillation, additional ethanol may be added. The organic phase of the distillate is returned to the reaction vessel. When the addition of methylamine is complete, the distillation is continued until no further water is formed. Then the alcohol is removed by fractional distillation under reduced pressure, the pressure being so selected that the temperature does not exceed 90° C. After cooling the reaction mixture to room temperature, the lower phase, which has a small volume, is separated off. If the concentration of 4,4,4-trifluoro-3-methylamino-crotonic acid ethyl ester in the solution is high, phase inversion may occur and the undesired phase is located in the upper region of the reaction vessel. After separating off the methylcyclohexane by fractional distillation of the organic phase in vacuo, 4,4,4-trifluoro-3-methylamino-crotonic acid ethyl ester is obtained in a yield of 98% of the theoretical yield.

What is claimed is:

1. A process for the preparation of a 4,4,4-trifluoro-3-methylamino-crotonic acid ester of formula I 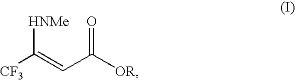
wherein
R is C₁-C₄alkyl, and
Me is methyl,
by dehydrating the corresponding trifluoroacetoacetic acid ester and methylamine in the presence of an inert reaction medium and of a C₁-C₄carboxylic acid, in which process the reaction is carried out at a temperature of from 75 to 100° C. without isolation of intermediates and the water that forms during the reaction is removed continuously from the reaction medium.

2. A process according to claim 1, wherein in formula I R is ethyl.

3. A process according to claim 1, wherein a water-immiscible reaction medium is used and the aqueous phase is separated from the reaction medium.

4. A process according to claim 1, wherein the appropriate alcohol is added to the reaction medium and the water is removed by distillation.

5. A process according to claim 1, wherein acetic acid is used as the C₁-C₄carboxylic acid.

6. A process according to claim 1, which is carried out at a temperature of from 80 to 85° C.

7. A process according to claim 1, wherein there is used as the reaction medium hexane, cyclohexane, methylcyclohexane, toluene, xylene, or a mixture thereof.

8. A process according to claim 1, wherein methylcyclohexane is used as the reaction medium.
